(19) United States
(12) Patent Application Publication
(10) Pub. No.: US 2016/0292381 A1
(43) Pub. Date: Oct. 6, 2016

(54) METHODS OF SIMULATING CHEMOTHERAPY FOR A PATIENT

(71) Applicant: HELOMICS CORPORATION, Pittsburgh, PA (US)

(72) Inventor: Nan SONG, Pittsburgh, PA (US)

(21) Appl. No.: 14/934,934

(22) Filed: Nov. 6, 2015

Related U.S. Application Data
(63) Continuation of application No. 14/668,088, filed on Mar. 25, 2015, now abandoned.
Application No. 14/668,088 is a continuation of application No. 13/127,337, filed on Jun. 9, 2011, now abandoned.
(60) Provisional application No. 61/110,730, filed on Nov. 3, 2008.

(51) Int. Cl. G06F 19/00; G01N 33/50

(52) U.S. Cl. CPC G06F 19/3437; G01N 33/5011; G01N 2800/52; G01N 2800/7028

(57) ABSTRACT

The present invention provides methods for predicting or modeling a chemotherapy outcome for a given patient The method produces chemoresponse data, and presents the chemoresponse data in a clinically meaningful context such that the data can be meaningfully interpreted and evaluated in a clinical context The method of the invention involves correlating in vitro chemoresponse results for a particular patient with historical treatment outcomes Where a population of historical outcomes are matched to the patient by one or more clinical vanables and such outcomes are matched to a potential treatment by the in vitro efficacy of the agent received, a meaningful simulation of the potential treatment for the patient can be constructed Simulations, such as survival curves, for a plurality of potential treatments may be generated and compared to contrast the estimated outcomes for several potential treatments, thereby providing the information desirable to design an individualized treatment regimen.

### CROSS-REFERENCE TO RELATED APPLICATIONS

This application claims priority to U.S. Provisional Application No. 61/110,730, filed Nov. 3, 2008 which is incorporated herein by reference in its entirety.

### FIELD OF THE INVENTION

The present invention relates generally to in vitro chemoresponse testing to assist physicians in the selection of chemotherapeutic agents for cancer patients on an individualized basis.

### BACKGROUND OF THE INVENTION

Traditionally, treatments for cancer patients are selected based on active agents and combinations identified to be most effective in large randomized clinical trials. However, since such therapy is not individualized, this approach often results in the administration of sub-optimal chemotherapy. The administration of sub-optimal or ineffective chemotherapy to a particular patient can lead to unsuccessful treatment, including death, disease progression, unnecessary toxicity, and higher health care costs.

In an attempt to individualize cancer treatment, in vitro drug-response assay systems (chemoresponse assays) have been developed to predict the potential efficacy of chemotherapy agents for a given patient prior to their administration. Although such in vitro systems are available, the use of these systems is not sufficiently widespread due, in-part, to difficulties in interpreting the data in a clinically meaningful way, as may be required in many instances to drive administration of an individualized treatment regimen. For example, while in vitro systems are recognized as predicting generally inactive and/or generally active agents, and/or for predicting short-term responses, such systems are not generally recognized as providing accurate estimations of patient survival with particular treatment regimens (Fruehauf et al., Endocrine-Related Cancer 9:171-182 (2002).

A chemoresponse assay providing readily interpretable results, including with respect to a panel of active agents having a range of activity against a patient's cells in vitro, would encourage or support a treating physician in administering an individualized treatment plan. Such a method could present a clear advantage of individualized treatments, as compared to non-individualized selection of agents based on large randomized trials.

### SUMMARY OF THE INVENTION

The present invention provides methods for predicting or estimating a chemotherapy outcome for a given cancer patient, to assist physicians in the selection of chemotherapeutic agents for individualized cancer treatment. The method produces chemoresponse data, and presents the chemoresponse data in a clinically meaningful context, such that the chemoresponse data can be meaningfully interpreted and evaluated to individualize patient treatment, as opposed to selecting conventional, non-individualized treatments for a patient's disease.

The method of the invention involves correlating in vitro chemoresponse results for a particular cancer patient in need of treatment, with historical in vitro chemoresponse data for which clinical treatment results or outcomes are known. Particularly, a patient's in vitro chemoresponse profile is compared to historical chemoresponse data having corresponding clinical outcomes, in which agents were found to produce (for example) a non-responsive, intermediate responsive, or responsive result in vitro, and were then selected for patient therapy.

In accordance with the invention, one can construct a meaningful model of treatment with a candidate chemotherapeutic agent on the basis of its in vitro efficacy against the patient's tumor cells. On the basis of this in vitro efficacy, a population of historical treatment outcomes may be selected and modeled to simulate the chemotherapy. The historical treatment outcomes each involved treatment with a chemotherapeutic agent, after that agent had demonstrated the selected level of in vitro efficacy. Such historical treatment outcomes are further matched to the patient by one or more clinical variables, such as, for example, cancer type and/or cancer stage. Relevant historical outcomes may be selected and used to generate a logistic regression or logistic model, or Cox model, to estimate survival or progression-free interval, or other outcome, for the patient upon receiving the candidate treatment. A plurality of such models, each providing a probability of an outcome for a candidate treatment, may be compared to contrast the estimated outcomes between different candidate agents. The invention thereby provides information to aid in designing an individualized treatment regimen. In certain embodiments, models for agents found to elicit a sensitive response against the patient's tumor cells in vitro are compared to models for agents found to elicit an intermediate response against the patient's tumor cells in vitro, to thereby estimate the difference in clinical benefit.

For example, the method comprises conducting chemoresponse testing with a panel of chemotherapeutic agents on cultured tumor cells from a patient. The tumor cells may be cultured from cohesive multicellular particulates (e.g., explants) of the patient's tumor specimen, so as to enrich for malignant cells and to provide sufficient cells representative of the tumor for testing in a short duration. The panel of chemotherapeutic agents are then graded for their in vitro efficacy on the cultured cells, e.g., as producing a responsive, intermediate responsive, or a non-responsive result. The chemotherapeutic agents may be graded for their in vitro efficacy using algorithms described herein. The in vitro efficacy grade for at least two agents in the panel are then each matched to a logistic or cox model for survival, progression-free interval, or other outcome, the models being generated from historical data. The historical data includes clinical outcome information for a historical patient population that each received a chemotherapeutic agent, after that agent had been used for in vitro chemoresponse testing.

Thus, the historical data to be modeled includes, most importantly, clinical treatment and outcome information with corresponding in vitro chemoresponse data. The historical data may include for each subject in the population: basic patient information, a description of clinical disease and the progression of the disease, in vitro chemoresponse data for one or a plurality of chemotherapeutic agents, the selected treatment regimen(s), and the patient's clinical outcome or response to treatment. The subject population for any given model or simulation may be selected on the basis of a plurality of disease variables, including (for example) cancer type, cancer stage, and debulking status, as well as the in vitro efficacy grade of the agent(s) received during therapy.

The differences between outcomes for two or more candidate agents (including combinations of agents) may be estimated, by comparing the models generated for the respective candidate agents. The comparison may determine, for example, a difference in the predicted survival, or probability of survival (or other event), upon treatment with each of the candidate agents. In certain embodiments, models are compared to contrast differences between the estimated efficacy of agents found to produce responsive and intermediate responsive results in vitro. Thus, the invention allows a physician or clinician to contrast the estimated clinical benefits of a plurality of candidate agents that each show some, albeit variable, level of activity against the patient's tumor cells in vitro.

### BRIEF DESCRIPTION OF THE FIGURES

FIG. 1 illustrates a Cox model showing that a responsive (R), intermediate responsive (IR), and non-responsive (NR) result in the ChemoFx® chemoresponse assay are correlative with progression free interval (A) and survival (B). Cancers were ovarian, fallopian tube, or peritoneal carcinoma.

FIG. 2 shows an exemplary Cox model for a subject population. The observed curve reflects the result of treatment (progression-free interval or survival) with agents found to be ineffective for the subjects in vitro (NR or non-responsive result in ChemoFx® assay), and where a more active alternative was available (IR or intermediate responsive). The simulated curve estimates the clinical outcome had the patients received the alternative drug. The simulated curve is based upon survival and progression-free interval data for a subject population that received, for therapy, a drug that produced an intermediate responsive result in vitro (with the ChemoFx® assay). The curves are based on subject populations matched for debulking status, cancer type, cancer stage, and primary versus recurrent cancer. Thus, the observed curve shows the probability of patient survival upon treatment with a drug that produces a non-responsive result in vitro, and demonstrates a survival time of about 8.1 months (point estimate). The simulated curve shows the probability of patient survival upon treatment with a drug that produces an intermediate responsive result in vitro, and models a survival time or progression-free interval of about 10.67 months.

FIG. 3 shows a survival curve (Cox model) based upon a single variant analysis using optimal and suboptimal debulking status.

FIG. 4 shows a survival curve (Cox model) based upon a single variant analysis using cancer stage (e.g., stage I-IIIA versus >IIIA).

FIG. 5 shows a survival curve (Cox model) based upon a single variant analysis with ChemoFx® result for the agent received during therapy. R is responsive, IR is intermediate responsive, and NR is non-responsive.

FIG. 6 shows a survival curve (Cox model) based upon a single variant analysis with alternate sensitive and intermediate sensitive treatments in the ChemoFx® assay.

FIG. 7 shows observed survival versus simulated survival for cohorts 2-6 (A). Cohorts 1-6 are described herein. For optimized cohorts 3 and 6 the observed and simulated are similar. For non-optimized cohorts 2, 4, and 5, the simulated time is larger than the observed time. (B) shows a Kaplan-Meier of observed and simulated data stratified by cohort. For cohorts 3 and 6, because the observed and simulated are so similar and overlay, the dotted lines are not visible. For optimized cohorts, the observed median and simulated median are identical. For non-optimized cohorts, these values differ by 22.8 months, 31.6 months, and 41.4 months, and represent the estimated survival of the patient had the patient received the alternate drug (see Table 2 below).

FIG. 8 shows a Kaplan Meier of observed and simulated data stratified by treatment. For treatments producing a non-responsive result in vitro the observed survival median was 41.4 months, while the simulated median for the alternative drug was 66.5 months. For treatments producing an intermediate responsive result in vitro the observed median survival was 63.0 months, while the simulated median with the alternative drug was 101.3 months.

FIG. 9 illustrates the use of historical data to estimate the outcome of treatment with candidate agents. First, historical data are selected or grouped based on defined variables. For example, the historical data may be grouped as to primary or recurrent cancer, and grouped on the basis of the in vitro efficacy of the agent received for therapy. As shown, there are six groups: (1) primary cancer and sensitive (S) in vitro efficacy, (2) primary cancer and intermediate (I) in vitro efficacy, (3) primary cancer and resistant (R) in vitro efficacy, (4) recurrent cancer and sensitive (S) in vitro efficacy, (5) recurrent cancer and intermediate (I) in vitro efficacy, and (6) recurrent cancer and resistant (R) in vitro efficacy. Using the corresponding clinical data (e.g., outcome of treatment), each of these groups may be used to build a model (e.g., Cox model), shown in FIG. 9 as survival curves. These curves may be compared to reflect differences in estimated clinical outcome between groups; for example, between group 1 (primary cancer and sensitive in vitro efficacy), and group 2 (primary cancer and intermediate in vitro efficacy).

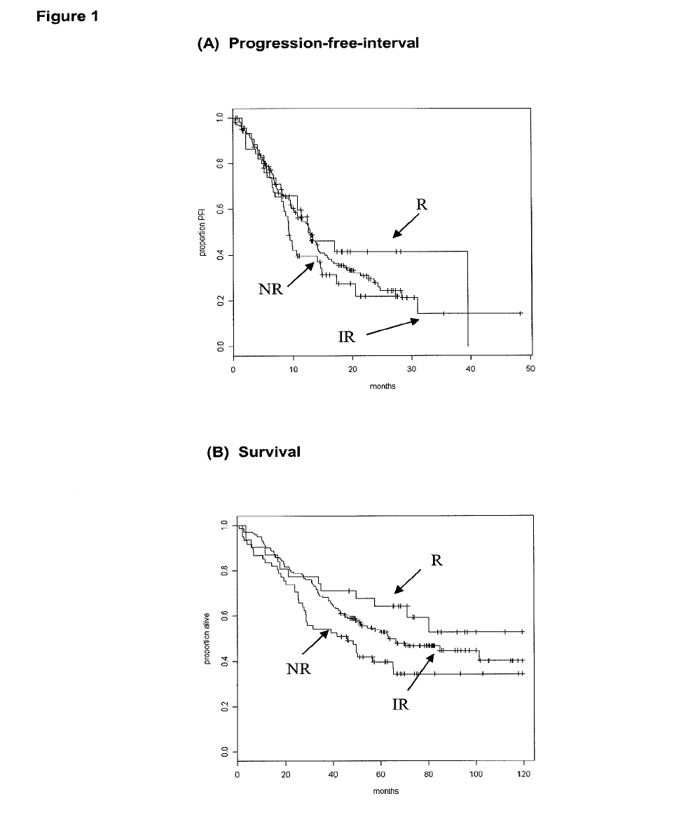

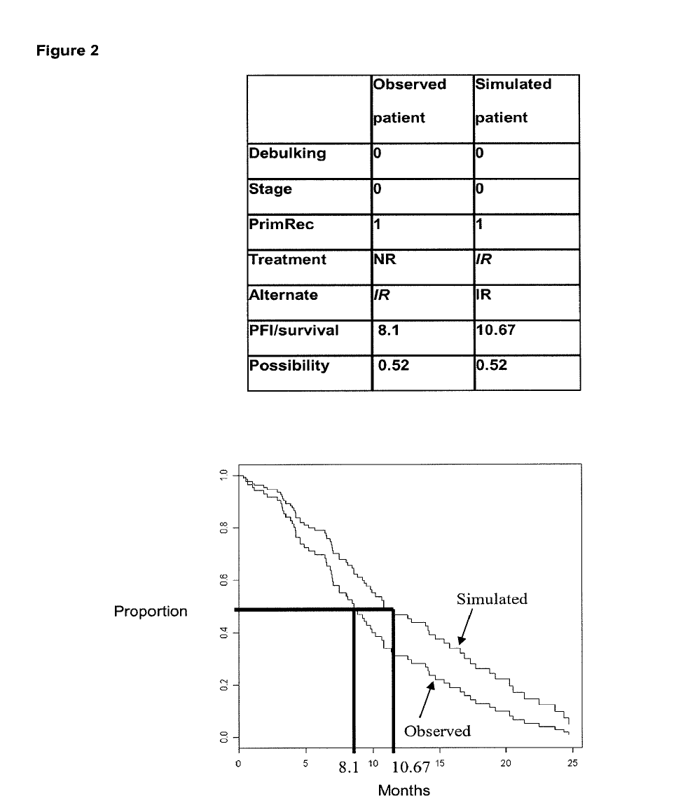

### DETAILED DESCRIPTION

The present invention provides methods for predicting or estimating a chemotherapy outcome for a given patient to assist physicians in the selection of chemotherapeutic agents for individualized cancer treatment. The method allows individualized treatment plans to be evaluated next to conventional treatments for a patient's disease, by presenting predicted or estimated outcomes of therapy. The method of the invention involves correlating in vitro chemoresponse results for a particular patient, with historical treatment data in which agents found to produce, for example, a non-responsive, intermediate responsive, or responsive result in vitro, were selected for therapy. A population of historical outcomes are matched to the patient by one or more clinical variables (including, for example, primary versus recurrent cancer), and such historical outcomes are matched to a potential treatment by the in vitro efficacy of the agent that was received for therapy. Thus, the invention simulates treatment(s) with agents that show variable efficacy grades against patient tumor cells in vitro, and allows meaningful comparisons of treatment(s) with agents showing responsive and intermediate responsive grades against the patient's tumor cells in vitro.

The method generally comprises conducting chemoresponse testing with a panel of chemotherapeutic agents on cultured tumor cells from a patient. The in vitro efficacy of each agent (including combinations) in the panel on the patient's cells in culture is graded, and two or more of these grades are matched to a simulated outcome (e.g., on the basis of their in vitro efficacy grades). For example, where an agent provides an intermediate responsive grade against a patient's cells in culture, therapy with this agent is modeled by selecting historical outcomes having certain defined clinical variables and involving treatment with a drug, after that drug produced an intermediate responsive grade in a chemoresponse test. Where an agent produces a responsive grade against a patient's cells in culture, therapy with this agent is matched to historical outcomes having certain defined clinical variables, and involving treatment with an agent, after that agent showed a responsive grade in a chemoresponse test. Historical outcomes are matched to the patient by a plurality of clinical variables, which are described herein. For example, where the patient has primary cancer with optimal debulking, historical outcomes may be selected that involved treatment for a primary cancer after optimal debulking.

Once matching outcomes are selected (e.g., from a database), the matching outcomes are modeled to estimate treatment of a patient matching the group criteria. The models may each take the form of a logistic model or Cox model. Two or more models may be compared to estimate a benefit of one potential therapy over another. The invention thereby estimates chemotherapy outcomes, such as survival, progression-free interval, or other outcome, so that effective chemotherapeutic agents may be distinguished from generally inactive agents and/or generally active agents effective for producing only short-term patient responses, and/or to present chemoresponse results in a clinically meaningful context.

### Chemoresponse Assay

The present invention involves conducting chemoresponse testing with a panel of chemotherapeutic agents on cultured cells from a cancer patient. The invention may be applicable to a variety of cancers, and exemplary cancer types include breast, ovarian, colorectal, endometrial, thyroid, nasopharynx, prostate, head and neck, liver, kidney, pancreas, bladder, brain, and lung. In certain embodiments, the tumor may be epithelial in nature, and/or may be a solid tissue tumor.

Several in vitro chemoresponse systems are known and art, and some are reviewed in Fruehauf et al., In vitro assay-assisted treatment selection for women with breast or ovarian cancer, Endocrine-Related Cancer 9: 171-82 (2002). In certain embodiments, the chemoresponse assay is as described in U.S. Pat. Nos. 5,728,541, 6,900,027, 6,887,680, 6,933,129, 6,416,967, 7,112,415, and 7,314,731 (all of which are hereby incorporated by reference in their entireties). The chemoresponse method may further employ the variations described in US Published Patent Application Nos. 2007/0059821 and 2008/0085519, both of which are hereby incorporated by reference in their entireties. Such chemoresponse methods are commercially available as the ChemoFx™ Assay (Precision Therapeutics, Inc, Pittsburgh, Pa.).

Briefly, in certain embodiments, cohesive multicellular particulates (explants) are prepared from a patient's tissue sample (e.g., a biopsy sample or surgical specimen) using mechanical fragmentation. This mechanical fragmentation of the explant may take place in a medium substantially free of enzymes that are capable of digesting the explant. Some enzymatic digestion may take place in certain embodiments. Generally, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³.

After the tissue sample has been minced, the particles are plated in culture flasks. The number of explants plated per flask may vary, for example, between one and 25, such as from 5 to 20 explants per flask. For example, about 9 explants may be plated per T-25 flask, and 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of a few weeks a cell monolayer will form. Further, it is believed (without any intention of being bound by the theory) that tumor cells grow out from the multicellular explant prior to stromal cells. Thus, by initially maintaining the tissue cells within the explant and removing the explant at a predetermined time (e.g., at about 10 to about 50 percent confluency, or at about 15 to about 25 percent confluency), growth of the tumor cells (as opposed to stromal cells) into a monolayer is facilitated. In certain embodiments, the tumor explant may be agitated to substantially release tumor cells from the tumor explant, and the released cells cultured to produce a cell culture monolayer. The use of this procedure to form a cell culture monolayer helps maximize the growth of representative tumor cells from the tissue sample.

Prior to the chemotherapy assay, the growth of the cells may be monitored, and data from periodic counting may be used to determine growth rates which may or may not be considered parallel to growth rates of the same cells in vivo in the patient. If growth rate cycles can be documented, for example, then dosing of certain active agents can be customized for the patient. Monolayer growth rate and/or cellular morphology may be monitored using, for example, a phase-contrast inverted microscope. Generally, the cells of the monolayer should be actively growing at the time the cells are suspended and plated for drug exposure. Thus, the monolayers will generally be non-confluent monolayers at the time the cells are suspended for drug exposure.

A panel of active agents may then be screened using the cultured cells. Generally, the agents are tested against the cultured cells using plates such as microtiter plates. For the chemosensitivity assay, a reproducible number of cells is delivered to a plurality of wells on one or more plates, preferably with an even distribution of cells throughout the wells. For example, cell suspensions are generally formed from the monolayer cells before substantial phenotypic drift of the tumor cell population occurs. The cell suspensions may be, without limitation, about 4,000 to 12,000 cells/ml, or may be about 4,000 to 9,000 cells/ml, or about 7,000 to 9,000 cells/ml. The individual wells for chemoresponse testing are inoculated with the cell suspension, with each well or “segregated site” containing about 10² to 10⁴ cells. The cells are generally cultured in the segregated sites for about 4 to about 30 hours prior to contact with an agent.

Each test well is then contacted with at least one pharmaceutical agent. The panel of chemotherapeutic agents may comprise at least one agent selected from a platinum-based drug, a taxane, a nitrogen mustard, a kinase inhibitor, a pyrimidine analog, a podophyllotoxin, an anthracycline, a monoclonal antibody, and a topoisomerase I inhibitor. For example, the panel may comprise 1, 2, 3, 4, or 5 agents selected from bevacizumab, capecitabine, carboplatin, cecetuximab, cisplatin, cyclophosphamide, docetaxel, doxorubicin, epirubicin, erlotinib, etoposide, 5-fluorouracil, gefitinib, gemcitabine, irinotecan, oxaliplatin, paclitaxel, panitumumab, tamoxifen, topotecan, and trastuzumab, in addition to other potential agents for treatment. In certain embodiments, the chemoresponse testing includes one or more combination treatments, such combination treatments including one or more agents described above. Generally, each agent in the panel is tested in the chemoresponse assay at a plurality of concentrations representing a range of expected extracellular fluid concentrations upon therapy.

Suitable pharmaceutical agents for use in accordance with the invention include those listed in the following table.

| Drug Name | Alternative Nomenclature |
|---|---|
| Altretamine | Hexalen ®, |
| | hydroxymethylpentamethylmelamine |
| | (HMPMM) |
| Bleomycin | Blenoxane ® |
| Carboplatin | Paraplatin ® |
| Carmustine | BCNU, BiCNU ® |
| Cisplatin | Platinol ®, CDDP |
| Cyclophosphamide | Cytoxan ®, Neosar ®, |
| | 4-hydroperoxycyclophosphamide, |
| | 4-HC |
| Docetaxel | Taxotere ®, D-Tax |
| Doxorubicin | Adriamycin ®, Rubex ®, Doxil ®* |
| Epirubicin | Ellence ® |
| Erlotinib | Tarceva ®, OSI-774 |
| Etoposide | VePesid ®, Etopophos ®, VP-16 |
| Fluorouracil | Adrucil ®, 5-FU, Efudex ®, Fluoroplex ®, |
| | Capecitabine*, Xeloda ®* |
| Gemcitabine | Gemzar ® |
| Ifosfamide | Ifex ®, 4-hydroperoxyifosfamide, 4-HI |
| Irinotecan/SN-38 | Camptosar ®, CPT-11, SN-38 |
| Leucovorin | Wellcovorin ® |
| Lomustine | CCNU, CeeNU ® |
| Melphalan | Alkeran ®, L-PAM |
| Mitomycin | Mutamycin ®, Mitozytrex ®, Mitomycin-C |
| Oxaliplatin | Eioxatin ® |
| Paclitaxel | Taxol ®, Abraxane ®* |
| Procarbazine | Matulane ®, PCZ |
| Temozolomide | Temodar ® |
| Topotecan | Hycamtin ® |
| Vinblastine | Velban ®, Exal ®, Velbe ®, Velsar ®, VLB |
| Vincristine | Oncovin ®, Vincasar PFS ®, VCR |
| Vinorelbine | Navelbine ®, NVB |

The efficacy of each agent in the panel is determined against the patient's cultured cells, by determining the viability of the cells (e.g., number of viable cells). For example, at predetermined intervals before, simultaneously with, or beginning immediately after, contact with each agent or combination, an automated cell imaging system may take images of the cells using one or more of visible light, UV light and fluorescent light. Alternatively, the cells may be imaged after about 25 to about 200 hours of contact with each treatment. The cells may be imaged once or multiple times, prior to or during contact with each treatment. Of course, any method for determining the viability of the cells may be used to assess the efficacy of each treatment in vitro.

In this manner the in vitro efficacy grade for each agent in the panel may be determined, for matching to historical outcomes. While any grading system may be employed, in certain embodiments the grading system may have from 2 or 3, to 10 response levels, e.g., about 3, 4, or 5 response levels. For example, when using three levels, the three grades may correspond to a responsive grade (e.g., sensitive), an intermediate responsive grade, and a non-responsive grade (e.g., resistant), as discussed more fully herein. In certain embodiments, the patient's cells show a heterogeneous response across the panel of agents, making the selection of an agent particularly crucial for the patient's treatment.

The chemoresponse assay described in this section may also be used to prepare the historical data, that is, once treatment outcomes can be documented. In this manner, a database of chemoresponse results with corresponding clinical variables and outcome determinations (as described herein) can be accumulated for modeling therapy for subsequent patients.

### Algorithms

The output of the assay is a series of dose-response curves for tumor cell survivals under the pressure of a single or combination of drugs, with multiple dose settings each (e.g., ten dose settings). To better quantify the assay results, the invention employs in some embodiments a scoring algorithm accommodating a dose-response curve. Specifically, the chemoresponse data are applied to an algorithm to quantify the chemoresponse assay results by determining an adjusted area under curve (aAUC).

However, since a dose-response curve only reflects the cell survival pattern in the presence of a certain tested drug, assays for different drugs and/or different cell types have their own specific cell survival pattern. Thus, dose response curves that share the same aAUC value may represent different drug effects on cell survival. Additional information may therefore be incorporated into the scoring of the assay. In particular, a factor or variable for a particular drug or drug class (such as those drugs and drug classes described) and/or reference scores may be incorporated into the algorithm.

For example, in certain embodiments, the invention quantifies and/or compares the in vitro sensitivity/resistance of cells to drugs having varying mechanisms of action, and thus, in some cases, different dose-response curve shapes. Exemplary drugs and drug classes are described herein at paragraphs [29] and [30]. In these embodiments, the invention compares the sensitivity of the patient's cultured cells to a plurality of agents that show some effect on the patient's cells in vitro (e.g., all score sensitive to some degree), so that the most effective agent may be selected for therapy. In such embodiments, an aAUC is calculated to take into account the shape of a dose response curve for any particular drug or drug class. The aAUC takes into account changes in cytotoxicity between dose points along a dose-response curve, and assigns weights relative to the degree of changes in cytotoxicity between dose points. For example, changes in cytotoxicity between dose points along a dose-response curve may be quantified by a local slope, and the local slopes weighted along the dose-response curve to emphasize cytotoxicity.

For example, aAUC may be calculated as follows.

Step 1: Calculate Cytotoxity Index (CI) for each dose, where CI=Mean_{drug}/Mean_{control}.

Step 2: Calculate local slope (S_{d}) at each dose point, for example, as S_{d}=(CI_{d}−CI_{d-1})/Unit of Dose, or S_{d}=(CI_{d-1}−CI_{d})/Unit of Dose.

Step 3: Calculate a slope weight at each dose point, e.g., W_{d}=1−S_{d}.

Step 4: Compute aAUC, where aAUC=ΣW_{d} CI_{d}, and where, d=1, 2, . . . , 10; aAUC ˜(0, 10); And at d=1, then CI_{d-1}=1. Equation 4 is the summary metric of a dose response curve and may used for subsequent regression over reference outcomes.

Usually, the dose-response curves vary dramatically around middle doses, not in lower or higher dose ranges. Thus, the algorithm in some embodiments need only determine the aAUC for a middle dose range, such as for example (where from 8 to 12 doses are experimentally determined, e.g., about 10 doses), the middle 4, 5, 6, or 8 doses are used to calculate aAUC. In this manner, a truncated dose-response curve might be more informative in outcome prediction by eliminating background noise.

The numerical aAUC value (e.g., test value) may then be evaluated for its effect on the patient's cells. For example, a plurality of drugs may be tested, and aAUC determined as above for each, to determine whether the patient's cells have a sensitive response, intermediate response, or resistant response to each drug.

In some embodiments, each drug is designated as, for example, sensitive, or resistant, or intermediate, by comparing the aAUC test value to one or more cut-off values for the particular drug (e.g., representing sensitive, resistant, and/or intermediate aAUC scores for that drug). The cut-off values for any particular drug may be set or determined in a variety of ways, for example, by determining the distribution of a clinical outcome within a range of corresponding aAUC reference scores. That is, a number of patient tumor specimens are tested for chemosenstivity/resistance (as described herein) to a particular drug prior to treatment, and aAUC quantified for each specimen. Then after clinical treatment with that drug, aAUC values that correspond to a clinical response (e.g., sensitive) and the absence of significant clinical response (e.g., resistant) are determined. Cut-off values may alternatively be determined from population response rates. For example, where a patient population is known to have a response rate of 30% for the tested drug, the cut-off values may be determined by assigning the top 30% of aAUC scores for that drug as sensitive. Further still, cut-off values may be determined by statistical measures.

In other embodiments, the aAUC scores may be adjusted for drug or drug class. For example, aAUC values for dose response curves may be regressed over a reference scoring algorithm adjusted for test drugs. The reference scoring algorithm may provide a categorical outcome, for example, sensitive (s), intermediate sensitive (i) and resistant (r), as already described. Logistic regression may be used to incorporate the different information, i.e., three outcome categories, into the scoring algorithm. However, regression can be extended to other forms, such as linear or generalized linear regression, depending on reference outcomes. The regression model may be fitted as the following: Log it (Pref)=α+β (aAUC)+γ (drugs), where γ is a covariate vector and the vector can be extended to clinical and genomic features. The score may be calculated as Score=β (aAUC)+γ (drugs). Since the score is a continuous variable, results may be classified into clinically relevant categories, i.e., sensitive (S), intermediate sensitive (I), and resistant (R), based on the distribution of a reference scoring category or maximized sensitivity and specificity relative to the reference.

The algorithms described in this section may also be used to prepare the historical data, that is, once treatment outcomes can be documented. In this manner, a database of chemoresponse results with corresponding clinical variables and outcome determinations (as described herein) can be accumulated for modeling therapy for subsequent patients.

### Clinical Variables

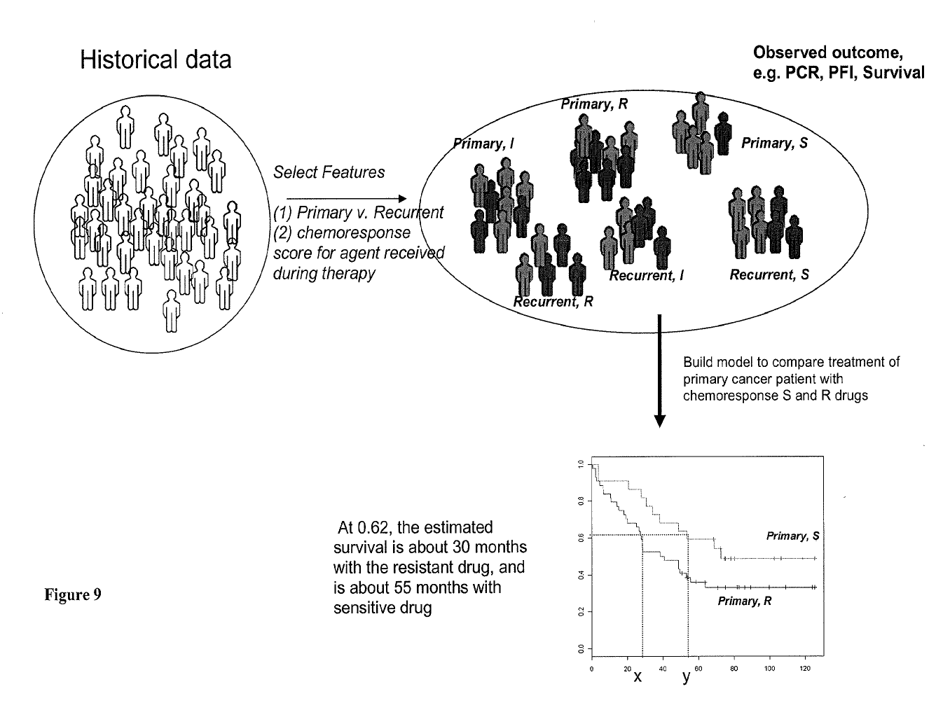

The in vitro efficacy of each agent in the panel on the patient's cells in culture is graded, and two or more of these grades are matched to historical data (e.g., on the basis of their in vitro efficacy grades), or matched to a model generated from historical data. For example, where an agent has a responsive grade for the patient's cells in culture, therapy with this agent is matched to historical outcomes in which a subject had received a drug for treatment that showed a responsive grade on the subject's tumor cells in culture. Where an agent has an intermediate responsive grade for the patient's cells in culture, therapy with this agent is matched to historical outcomes (e.g., as stored in a database) in which a subject had received a drug (or a similar drug) for treatment that showed an intermediate responsive grade on the subject's tumor cells in culture. See FIG. 9. Such historical outcomes are also matched to the patient by a plurality of clinical variables, as described in detail below.

Thus, the invention generally employs a database of historical data, and which may comprise for each of a plurality of patients: basic patient information (e.g., age, sex, performance status, etc.); clinical description of the patient's disease (e.g., cancer type, cancer stage, cancer grade, tumor histology, debulking status, level of tumor or serum marker(s), extent and duration of remission, etc.); selected treatment regimen(s); the patient's response to the treatment(s) including treatment outcomes; disease progression during and after treatment; corresponding in vitro chemoresponse data for the agent(s) received during therapy, and potentially other agents; and the outcome of cancer treatment, such as duration of survival or progression free interval from initiation of treatment or from diagnosis. Such information (which is described further below) may be stored on a computer readable medium in a retrievable and searchable manner, so as to select matching subjects and prepare a model or simulated outcome from the selected population.

Thus, the patient is matched to historical outcomes by one or more clinical variables, including one or more of cancer type, cancer stage, cancer grade, tumor debulking status, the presence, absence, or level of one or more tumor markers, primary versus recurrent cancer, interval of relapse for recurrent cancer patient, tumor histology, patient age, investigational site, number and/or type of prior drug treatments, an in vitro chemoresponse profile, time since diagnosis, patient's performance status, and extent of remission. In certain embodiments, the clinical variables include at least primary versus recurrent cancer, cancer stage, and debulking status. While these variables may be scored by any means known in the art, in certain embodiments, the clinical variables may be scored as described below.

The subject population may be matched to the patient on the basis of debulking status prior to chemotherapy. In this context, debulking status means the reduction of tumor size due to surgery or radiation treatment. Debulking status may be scored categorically, for example, as optimal or sub-optimal. For example, an optimal score may include patients in which the residual disease after radiation and/or surgery was 5 about 1 cm. A suboptimal score may include patients in which the residual disease after radiation and/or surgery was greater than about 1 cm.

The subject population may be matched to the patient on the basis of cancer type, for example, breast, ovarian, colorectal, endometrial, thyroid, nasopharynx, prostate, head and neck, liver, kidney, pancreas, bladder, brain, and lung. In some embodiments, cancer type is classified broadly, e.g., gynecological cancer. Alternatively, or in addition, the cancer may be classified by tumor histology, for example, using the classification system described in ROBBINS BASIC PATHOLOGY (Eighth Edition), or other system known in the art. In some embodiments, the tumor histology of the patient may be classified, and used to select outcomes from the available clinical data, by any of the following histologic epithelial cell types: serous adenocarcinoma, endometroid adenocarcinoma, mucinous adenocarcinoma, undifferentiated adenocarcinoma, transitional cell adenocarcinoma, or adenocarcinoma. Thus, such histological characterization of the patient's tumor, may, in some embodiments, be used to match outcomes to the patient, optionally in addition to classification by cancer type and stage.

Systems of cancer staging, which may be used to classify patients and subjects, are known in the art, and such systems may differ between cancer types. Such systems include TNM, FIGO, Roman Numeral Staging, Dukes Staging system, among others. Any system of cancer staging known in the art may be employed in accordance with the invention.

TNM Staging is used for solid tumors, and is an acronym for the words “Tumor”, “Nodes”, and “Metastases”. Each of these criteria is separately listed and paired with a number to indicate the TNM stage. For example, a T1N2M0 cancer is a cancer with a T1 tumor, N2 involvement of the lymph nodes, and no metastases (no spreading through the body). Tumor (T) refers to the primary tumor and carries a number of 0 to 4. N represents regional lymph node involvement and can also be ranked from 0 to 4. Metastasis is represented by the letter M, and is 0 if no metastasis has occurred, or else 1 if metastases are present. Within the TNM system, a cancer may also be designated as recurrent, meaning that it has appeared again after being in remission or after all visible tumor has been eliminated. Recurrence can either be local, meaning that it appears in the same location as the original, or distant, meaning that it appears in a different part of the body. The TNM system may be employed for cancer such as breast cancer, lung cancer, kidney cancer, prostate cancer, bladder cancer, colon cancer, melanoma, cancer of the larynx, cervical, and ovarian.

Gynecological cancers, such as cervical, ovarian, and vaginal cancers may employ the FIGO staging system (International Federation of Gynecology and Obstetrics), or similar system. This system classifies the diseases in Stages 0 through IV depending on the extent of the tumor (T), whether the cancer has spread to lymph nodes (N) and whether it has spread to distant sites. The definition of T, N and M is as follows. Tumor Extent (T) may be scored as: Tis, the cancer is not invading into the underlying tissues; T1, the cancer is only in the vagina; T2, the cancer has grown through the vaginal wall, but not as far as the pelvic wall; T3, the cancer is growing into the pelvic wall; T4, the cancer is growing into the bladder or rectum. Lymph Node Spread of Cancer (N) may be scored as: N0, no lymph node spread; N1, spread to lymph nodes in the pelvis or groin. Distant Spread of Cancer (M) is scored as: M0, no distant spread; or M1, the cancer has spread to distant sites. In Stage 0 (T1s, N0, M0), cancer cells are limited to the epithelium (lining layer) of the vagina and have not spread to other layers of the vagina. In Stage I (T1, N0, M0), the cancer has invaded (spread beneath) the epithelium but is confined to the vaginal mucosa (lining). In Stage II (T2, N0, M0), the cancer has spread to the connective tissues next to the vagina but has not spread to the wall of the pelvis, to other organs, or to lymph nodes. In Stage III (T1,2, N1, M0; T3, N0,1, M0), cancer extends to the wall of the pelvis and/or has spread to lymph nodes. In Stage IVA (T4, Any N, M0), cancer has spread to organs next to the vagina (such as the bladder or rectum). It may or may not have spread to lymph nodes. In Stage IVB (Any T, Any N, M1), cancer has spread to distant organs such as the lungs.

Overall Stage Grouping is also referred to as Roman Numeral Staging. This system uses numerals I, II, III, and IV (plus the 0) to describe the progression of cancer. For illustration, using the overall stage grouping, Stage I cancers are localized to one part of the body; Stage II cancers are locally advanced, as are Stage III cancers. Whether a cancer is designated as Stage II or Stage III can depend on the specific type of cancer; for example, in Hodgkin's Disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized, or spread to other organs or throughout the body. This system may be employed with, for example, liver cancer, among others.

In some embodiments, the subject population may be matched with the patient on the basis of performance status (e.g., at a similar time during the disease course, such as at about the time of diagnosis or at about the time treatment is initiated). Performance status quantifies cancer patients' general well-being. Methods for scoring a patient's performance status are known in the art. For example, this measure is used to determine whether a patient can receive chemotherapy, whether dose adjustment is necessary, and as a measure for the required intensity of palliative care. It is also used in oncological randomized controlled trials as a measure of quality of life. There are various scoring systems, including the Karnofsky score and the Zubrod score. Parallel scoring systems include the Global Assessment of Functioning (GAF) score, which has been incorporated as the fifth axis of the Diagnostic and Statistical Manual (DSM) of psychiatry. The Karnofsky score runs from 100 to 0, where 100 is “perfect” health and 0 is death. The score may be employed at intervals of 10, where: 100% is normal, no complaints, no signs of disease; 90% is capable of normal activity, few symptoms or signs of disease, 80% is normal activity with some difficulty, some symptoms or signs; 70% is caring for self, not capable of normal activity or work; 60% is requiring some help, can take care of most personal requirements; 50% requires help often, requires frequent medical care; 40% is disabled, requires special care and help; 30% is severely disabled, hospital admission indicated but no risk of death; 20% is very ill, urgently requiring admission, requires supportive measures or treatment; and 10% is moribund, rapidly progressive fatal disease processes.

ECOG scoring system for performance status includes: 0, fully active, able to carry on all pre-disease performance without restriction; 1, restricted in physically strenuous activity but ambulatory and able to carry out work of a light or sedentary nature, e.g., light house work, office work; 2, ambulatory and capable of all selfcare but unable to carry out any work activities, up and about more than 50% of waking hours; 3, capable of only limited selfcare, confined to bed or chair more than 50% of waking hours; 4, completely disabled, cannot carry on any selfcare, totally confined to bed or chair; 5, dead.

In some embodiments, the patient's disease is primary cancer, and the subjects are matched to the patient for pre-treatment performance status. In other embodiments, the patient's disease is recurrent, and the patient's performance status is matched with a subject population having the same performance status at recurrence.

Additional clinical variables, that may be quantified in cultured tumor cells or patient samples as appropriate, include the presence, absence, or level of certain tumor markers, including secreted factors and cell surface markers, and the level of circulating tumor cells or tumor-associated RNA or DNA. Exemplary markers include the overexpression of Her-2 (e.g., for breast cancer) on cultured tumor cells, level of PSA in patient serum (e.g., in the case of prostate cancer), the level of Nuclear Matrix Protein in urine, and carcinoembryonic antigen (CEA) serum levels. For example, such markers may be assayed in appropriate samples by, e.g., Western blot, dot blot, immunoprecipitation, ELISA, or immunohistochemistry for protein markers, and oligonucleotide arrays or quantitative PCR for RNA markers. These and other functionally equivalent assays may allow measurement of quantitative differences in expression, size, or state (e.g. oxidative state or phosphorylation state), or differences in cellular localization associated with cancerous phenotype or associated with response to chemotherapy or other drug treatment. Other assays known to those skilled in the art may be used to detect and/or to quantify such markers.

The patients may further be classified by the secretion of one or more markers of angiogenesis or tumor aggressiveness/invasiveness. For example, the clinical variables may include at least one angiogenesis-related factor selected from VEGFNPF, bFGF/FGF-2, IL-8/CXCL8, EGF, Flt-3 ligand, PDGF-AA, PDGF-AA/BB, IP-10/CXCL10, TGF-β1, TGF-β2, and TGF-β3. Such markers may be as described in PCT/US08/58001, which is hereby incorporated by reference, and may be determined in cultured tumor cells (e.g., in parallel with the chemoresponse assay), or may be otherwise determined in patient samples (e.g., blood/serum samples).

In certain embodiments, historical outcomes are matched to the patient (or potential treatment) by the agent, or class of the agent, received. That is, where doxorubicin is a candidate agent for a particular patient, historical outcomes may be selected where doxorubicin, or a similar agent, was administered to the subjects (and in vitro efficacy results for doxorubicin or similar agent are available for the subject). For the purpose of matching clinical agents, agents may be classified on the basis of biological target, known response profiles, mechanism of action, or chemical structure. For example, agents may be classified as a platinum-based drug, a taxane, a nitrogen mustard, a kinase inhibitor, a pyrimidine analog, a podophyllotoxin, an anthracycline, a monoclonal antibody (or monoclonal antibody against a particular target), and a topoisomerase I inhibitor. Thus, where a candidate agent for the patient is a taxane, outcomes in which a taxane was administered are selected for simulating an outcome for the patient's treatment with the taxane.

In certain embodiments, subjects are selected from the database for modeling chemotherapy by an in vitro chemoresponse profile. That is, subjects are selected based on their in vitro efficacy profile for at least two agents (e.g., 2, 3, or 4 agents), at least one of which the patient received for therapy. For example, where the patient's tumor cells have shown to be responsive to agent A in vitro, and intermediate responsive to an agent B in vitro, subjects are matched to the patient for this same profile of responsiveness with agents A and B.

In these and other embodiments, the patients are matched to the subject population by the extent of remission prior to treatment. For example, patients and subjects may be scored as having a complete remission (e.g., disease disappears), partial remission (e.g., disease shrinks), stable remission (e.g., disease does not progress), and no remission (e.g., disease progression).

In some embodiments, the patient and the subjects are not pan-responsive or pan-non-responsive with the in vitro chemoresponse testing, that is, the patient and the subjects each show a varied response to a panel of agents in vitro.

### Modeling Outcomes

After a patient's specimen has been cultured, and in vitro efficacy results obtained against a panel of agents, and after historical outcomes matching the patient's profile have been selected for at least two candidate agents in the panel, a model is constructed to simulate therapy with the candidate agents. The model may be a logistic or cox model, for example.

Generally, a Cox model consists of two parts: the underlying hazard function, describing how hazard (risk) changes over time, and the effect parameters, describing how hazard relates to other factors. The proportional hazards assumption is the assumption that effect parameters multiply hazard: for example, if taking drug X halves your hazard at time 0, it also halves your hazard at time 1, or time 0.5, or time t for any value of t. The effect parameter(s) estimated by any proportional hazards model can be reported as hazard ratios.

For example, a Cox model may estimate the hazard (or risk) of death, or other event of interest, for individuals given their prognostic variables. In one embodiment, a Cox model may specify the hazard ratio for an individual as: λᵢ(t)=λ₀(t)e^{x}^{ᵢ}^{(t)β}

The simulated outcome may take the form of Kaplan-Meier estimator (also known as the product limit estimator), estimating a survival function for example. A plot of the Kaplan-Meier estimate of the survival function is a series of horizontal steps of declining magnitude which, when a large enough sample is taken, approaches the true survival function for that population. The value of the survival function between successive distinct sampled observations (“clicks”) is assumed to be constant.

Alternatively, the matched historical outcomes may be selected and used to generate a logistic model (e.g., logistic regression), to estimate the probability of an outcome.

The goal of logistic regression is to correctly predict the category of outcome for individual cases using the most parsimonious model. The output for logistic regression is generally categorical, such as 0 or 1, while the output for KM or Cox model is continuous, as KM and Cox models take censor information into account. Logistic regression is a model for predicting the probability of occurrence of an event by fitting data to a logistic curve. It makes use of several predictor variables that may be either numerical or categorical. For example, the probability that a person has a heart attack within a specified time period might be predicted from knowledge of the person's age, sex and body mass index.

The outcome to be modeled, whether a logistic or Cox model is employed, may be an objective response, a clinical response, or a pathological response to treatment. The outcome may be determined based upon the techniques for evaluating response to treatment of solid tumors as described in Therasse et al., New Guidelines to Evaluate the Response to Treatment in Solid Tumors, J. of the National Cancer Institute 92(3):205-207 (2000), which is hereby incorporated by reference in its entirety. For example, the outcome may be survival, progression-free interval, or survival after recurrence. The timing or duration of such events may be determined from about the time of diagnosis or from about the time treatment (e.g., chemotherapy) is initiated. Alternatively, the outcome may be based upon a reduction in tumor size, tumor volume, or tumor metabolism, or based upon overall tumor burden, or based upon levels of serum markers especially where elevated in the disease state (e.g., PSA). The outcome in some embodiments may be characterized as a complete response, a partial response, stable disease, and progressive disease, as these terms are understood in the art.

In certain embodiments, the outcome is a pathological complete response. A pathological complete response, e.g., as determined by a pathologist following examination of tissue (e.g., breast or nodes in the case of breast cancer) removed at the time of surgery, generally refers to an absence of histological evidence of invasive tumor cells in the surgical specimen.

Simulations, as described above, for a plurality of potential treatments may be generated and compared to contrast the estimated outcomes for several potential treatments, thereby providing the information desirable to design an individualized treatment regimen.

Methods for comparing and contrasting simulations (e.g., routine tests for analyzing censured data) are known in the art, and include log-rank test, Wilcoxin test, or −2 log R. In certain embodiments, at least two agents in a patient's panel are selected, and matched to historical outcomes as described, where a first agent has a responsive in vitro efficacy grade, and a second agent has a non-responsive or intermediate responsive in vitro efficacy grade. Alternatively, or in addition, the first agent has a responsive or intermediate responsive in vitro efficacy grade, and the second agent has a non-responsive in vitro efficacy grade. Such curves are compared (e.g., by log-rank test) to determine the estimated difference in outcome between treatment with a responsive agent, intermediate responsive agent, and/or a non-responsive agent.

In comparing the models and/or curves generated for the candidate agents, estimated outcomes may be inferred from each model or curve. The estimated outcomes may reflect mean or median outcomes (e.g., mean or median survival), or may reflect a probability of an outcome (e.g., probability of survival or progression-free interval for a particular duration). In some embodiments, a “personalized number” is generated to further identify a particular patient's place on the model or curve. For example, the personalized number may be generated on the basis of the patient's genomic signature, gene expression levels, and/or serum marker levels.

Such information as described herein may be provided to a treating physician as a report to aid chemotherapy selection for the patient.

The invention will be further illustrated by the following Examples.

### Examples

A retrospective multi-institutional study was conducted to determine survival outcomes for patients with advanced ovarian, fallopian tube, or peritoneal carcinoma whose physicians had ordered the ChemoFx® assay (Precision Therapeutics, Inc., Pittsburgh, Pa.). Patients who met the following inclusion criteria were considered eligible for this analysis: 1) diagnosis of primary ovarian, fallopian tube, or peritoneal carcinoma, 2) the patient was treated with at least one cycle of a drug for which a ChemoFx® assay result was available, 3) FIGO (International Federation of Gynecology and Obstetrics) Stage II-IV disease, one of the following histologic epithelial cell types: serous adenocarcinoma, endometroid adenocarcinoma, mucinous adenocarcinoma, undifferentiated adenocarcinoma, transitional cell adenocarcinoma, or adenocarcinoma—not otherwise specified (N.O.S.). Progression free survival data from a subset of these patients (n=179) had been previously published.

Selection of treatment was at the discretion of the treating physician. In some cases, the physician may have used the assay to assist in the choice of therapy. Chemotherapy was administered from Jul. 1, 1997 through Dec. 1, 2003.

The Social Security Death Index was used to ascertain survival information. All patients who had not died were confirmed to be alive as of Jul. 12, 2007, which serves as the censoring date for this analysis. Survival was calculated from the earliest date of initiation of chemotherapy (Jul. 1, 1997) to date of documented death.

### Chemoresponse Assay

Specimens from surgically-excised ovarian carcinomas were submitted for testing with the ChemoFx® Assay. Briefly, primary cultures of cells were grown from the submitted specimens and incubated with a panel of therapeutic drugs selected by the referring physician. Six different drug concentrations were tested for each chemotherapeutic agent, representing the range of extracellular fluid concentrations expected during typical therapy, as well as sub- and supra-therapeutic levels. The percentages of cells remaining after drug treatment were used to construct dose-response curves. Each dose-response curve was reviewed and scored using a numeric system from 0 to 5. The score was based on the number of doses that resulted in ≧35% reduction in the total surviving cell fraction. The concentrations at which the threshold of cell reduction was noted determined the numerical score. For the purposes of this investigation, assay score results were classified as non-responsive (score of 0), intermediate responsive (score of 1-3), or responsive (score of 4-5).

### Estimating Survival

Overall survival (OS) was defined as the time from date of initiation of chemotherapy to date of death. OS rates were estimated by the Kaplan-Meier method and the differences between patients who received a drug to which they tested non-responsive (NR), intermediate-responsive (IR), and responsive (R) were compared by the log-rank test. Univariate and multivariate Cox proportional hazard models were used to evaluate the correlation of OS with the ChemoFx® assay. The multivariate model was selected by a backwards stepwise method. A P value of less than or equal to 0.05 was considered statistically significant. Statistical analysis was performed using Statistical Analysis System (SAS) version 8.1 (SAS Institute, Cary, N.C.) and R 2.4 (The R Foundation for Statistical Computing, Vienna, Austria).

The chemotherapy drugs tested on each tumor and the chemotherapy administered to the patient were chosen by the treating physician. As a result, a considerable number of patients were treated with combination chemotherapy even though only individual agents were tested. To score tests when an exact match was absent, the single-agent score was used in the following hierarchy (based upon relative efficacy, namely, the clinical literature response rate) (most to least): platinum, taxanes, cyclophosphamide, doxorubicin, and then fluorouracil (5FU). For example, if a patient received carboplatin/taxol combination chemotherapy but did not have this combination tested in the ChemoFx® assay, the score for carboplatin was used if performed, and if carboplatin was not tested, the taxanes score was used. Only single agents found in the administered combination were used for matching.

As the majority of patients received platinum based chemotherapy, it is impossible to directly evaluate the response of these patients to alternative therapies identified by the assay. In order to determine whether the assay results for multiple drugs was simply prognostic of response to chemotherapy in general, rather than predictive of response to specific agents, we compared survival analyses among only those patients who demonstrated a heterogeneous response to the tested agents. For the purpose of this analysis, patients were categorized into the following 3 groups: 1) pan-nonresponsive, 2) pan-responsive, and 3) heterogeneously responsive. A patient was considered pan-nonresponsive if the tumor had a ChemoFx® assay score of 0 for the entire range of drugs tested; a patient was considered pan-responsive if the tumor had the same ChemoFx® assay score, e.g., a score of 1, 2, 3, 4, or 5, for all the drugs tested. Patients were considered heterogeneous where tumors demonstrated a variable pattern of response. OS rates were compared by the Kaplan-Meier method and the differences between patients were calculated by log-rank tests.

The chemotherapeutic agent a patient received was determined by the treating physician. In some instances, there were agents in the panel assayed to which the patient tested more responsive than to the agent the patient actually received. To simulate how patients in this situation might have performed had they received an agent to which they were considered more responsive (if one existed) than the agent the patient received, a prediction model was created. Patients were grouped into 6 cohorts as shown in Table 1. Cohorts 1, 3, and 6 were considered optimized because none of the drugs tested were considered by the assay to be more likely to generate a patient response than the drug the patient actually received. Cohorts 2, 4, and 5 were considered non-optimized because in those cohorts the assay predicted greater tumor sensitivity for drugs other than the drug received.

TABLE 1
Cohort compositions

| Cohort | Treatment Drug | Alternate Drug |
|---|---|---|
| 1 | NR | NR |
| 2 | NR | IR |
| 3 | IR | IR |
| 4 | NR | R |
| 5 | IR | R |
| 6 | R | R |

NR is non-responsive, IR is intermediate responsive, and R is responsive.

The prediction model was generated as follows. Based on the outcomes of patients in the optimized cohorts 1, 3, and 6, a model to predict patient outcome was generated based on the available clinical factors. More particularly, for PFI, primary/recurrent, debulking, and stage were included as clinical variables. For survival analysis, since all patients were primary, only debulking and stage were included as clinical variables. For each patient in the non-optimized cohorts 2, 4, and 5, using their individual covariates, a simulated OS time was determined by using the model generated on the optimized cohorts (Cohort 3 for Cohort 2, and Cohort 6 for Cohorts 4 and 5). Optimized survival estimates were then calculated for the patients in Cohorts 2, 4, and 5, based on their simulated survival time by the Kaplan Meier method.

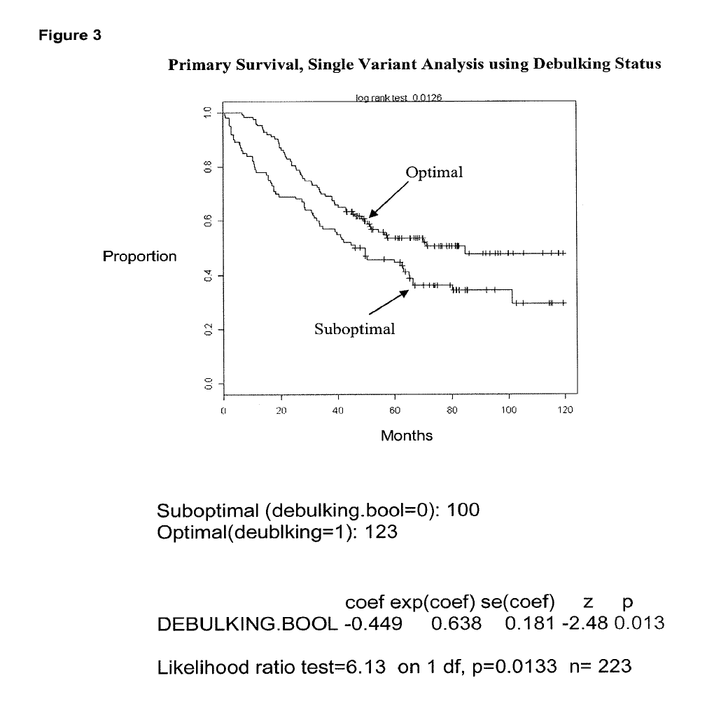

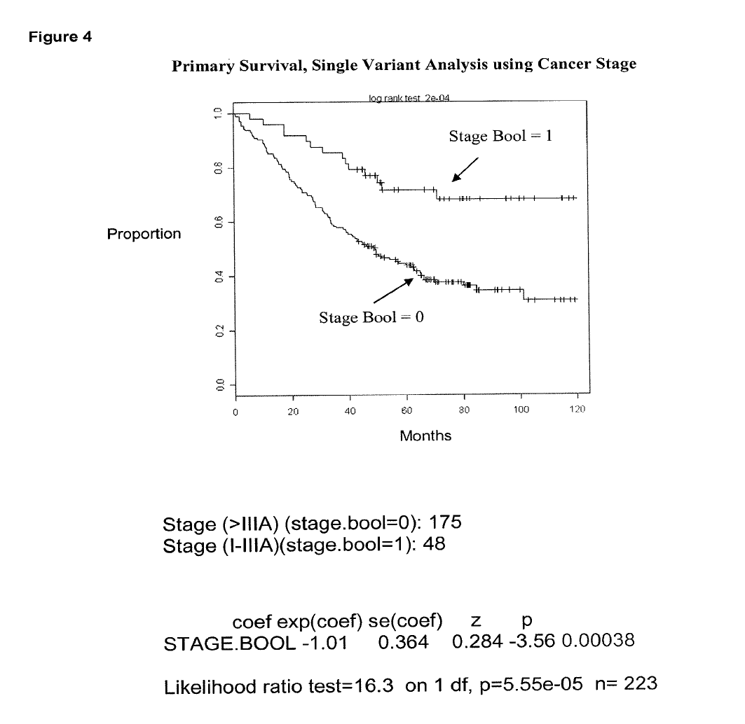

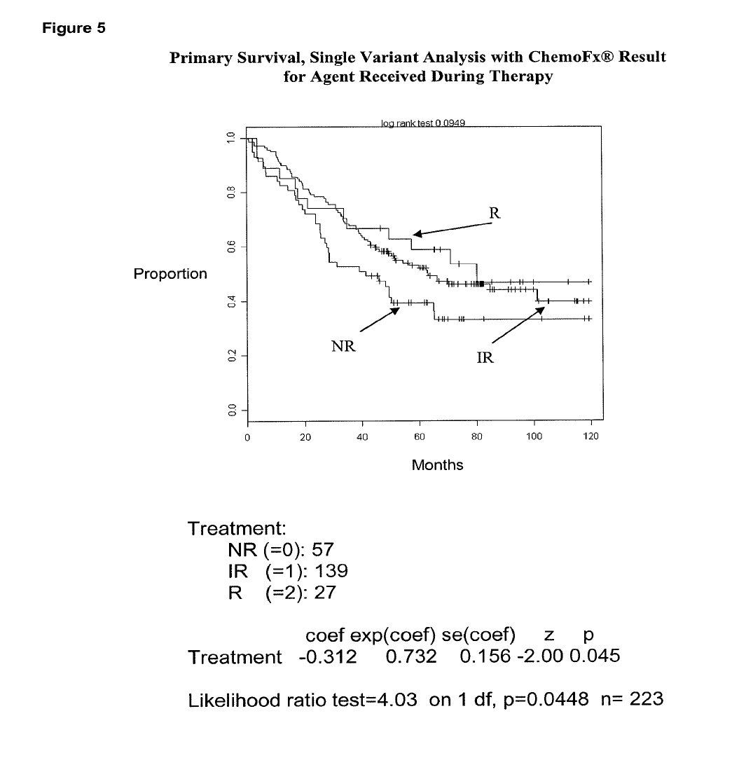

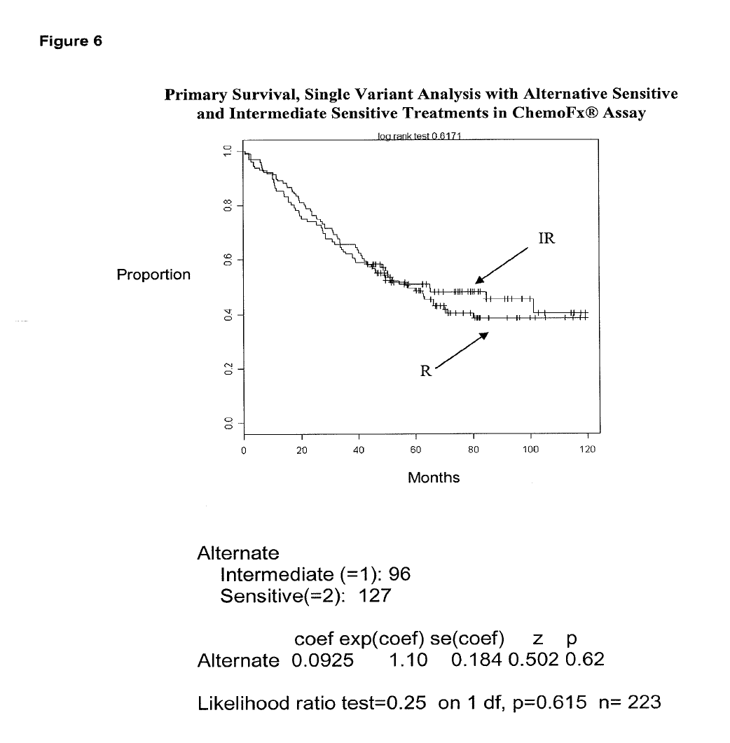

FIGS. 3-6 show single variant correlations with debulking status, cancer stage (classified as stages I-IIIA or >IIIA), in vitro efficacy of the drug received (classified as R, IR, and NR), and alternative treatments with intermediate responsive or responsive grades in culture. A summary of the single variant analyses is as follows:

| | coef | exp(coef) | se(coef) | z | p |
|---|---|---|---|---|---|
| DEBULKING.BOOL | −0.449 | 0.638 | 0.181 | −2.48 | 0.013 |
| STAGE.BOOL | −1.01 | 0.364 | 0.284 | −3.56 | 0.00038 |
| Treatment | −0.312 | 0.732 | 0.156 | −2.00 | 0.045 |
| Alternate | 0.0925 | 1.10 | 0.184 | 0.502 | 0.62 |

A summary of the multivariate analyses is as follows:

| | coef | exp(coef) | se(coef) | z | p |
|---|---|---|---|---|---|
| DEBULKING.BOOL | −0.289 | 0.749 | 0.184 | −1.57 | 0.1200 |
| STAGE.BOOL | −0.928 | 0.396 | 0.289 | −3.21 | 0.0013 |
| Treatment | −0.376 | 0.687 | 0.167 | −2.25 | 0.0240 |
| Alternate | 0.204 | 1.226 | 0.201 | 1.01 | 0.3100 |

Likelihood ratio test=24.2 on 4 df, p=7.36e-05 n=223

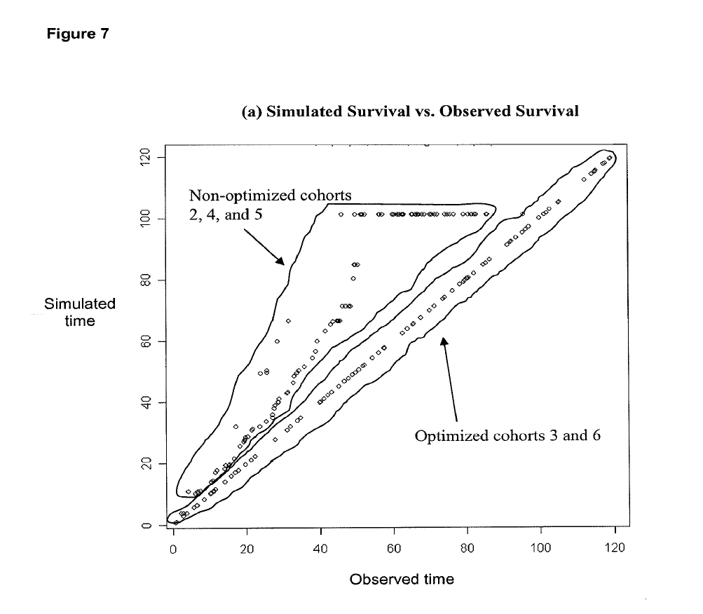

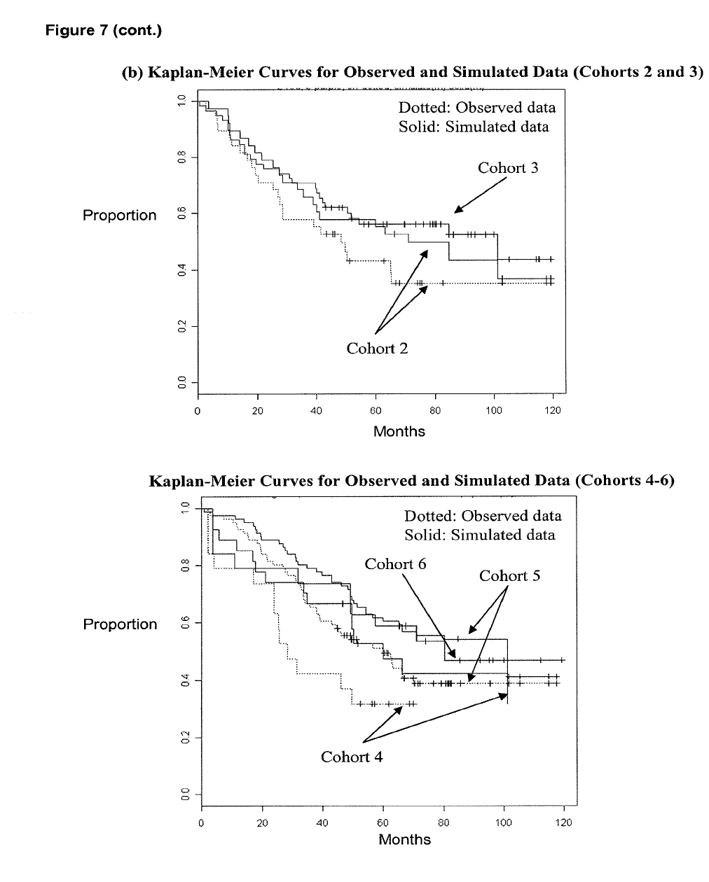

FIG. 7A shows observed survival versus simulated survival for cohorts 2-6. For optimized cohorts 3 and 6 the observed and simulated curves are similar. For non-optimized cohorts 2, 4, and 5, the simulated time is larger than the observed time. FIG. 7B shows a Kaplan-Meier curve of observed and simulated data stratified by cohort. For cohorts 3 and 6, because the observed and simulated are so similar and overlay, the dotted lines are not visible. For optimized cohorts, the observed median and simulated median are identical. For non-optimized cohorts, these values differ by 22.8 months, 31.6 months, and 41.4 months, and represent the estimated survival of the patient had the patient received the alternate drug. The results are summarized in Table 2.

TABLE 2
Differences between Observed and Simulated Medians

| | | Observed | Simulated | |
|---|---|---|---|---|
| Cohort | N | Median | median | Diff |
| 1 | 2 | 61.2 | | |
| 2 | 38 | 48.4 | 71.2 | 22.8 |
| 3 | 58 | 101.3 | 101.3 | |
| 4 | 19 | 28.3 | 59.9 | 31.6 |
| 5 | 81 | 59.9 | 101.3 | 41.4 |
| 6 | 27 | 80.4 | 80.4 | |

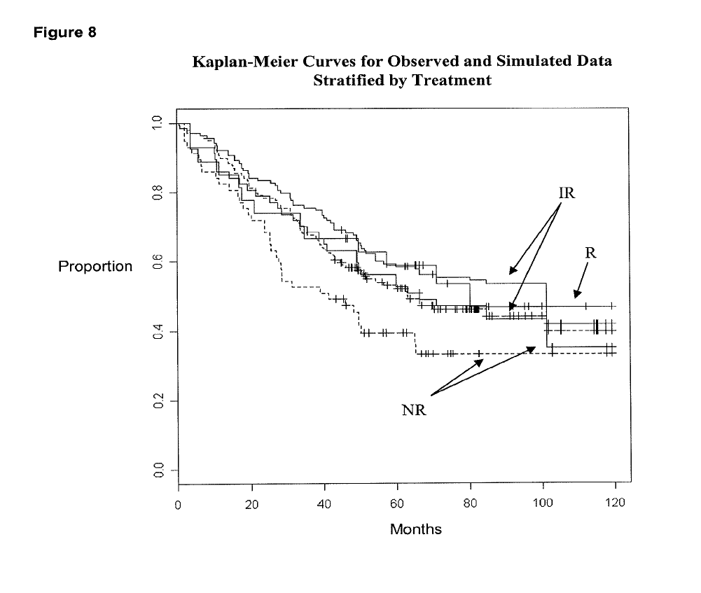

FIG. 8 shows a Kaplan-Meier curve of observed and simulated data stratified by treatment. For treatments producing a non-responsive result in vitro the observed survival median was 41.4 months, while the simulated median for the alternative drug was 66.5 months. For treatments producing an intermediate responsive result in vitro the observed median survival was 63.0 months, while the simulated median with the alternative drug was 101.3 months (see Table 3).

TABLE 3
Median for Observed and Simulated Data Stratified by Treatment

| | | Observed | Simulated |
|---|---|---|---|
| Treatment | N | Median | median |
| NR | 57 | 41.4 | 66.5 |
| IR | 139 | 63.0 | 101.3 |
| R | 27 | 80.4 | 80.4 |

### Use of Historical Data to Model Treatment Alternative

FIG. 9 illustrates the use of historical data to model treatment alternatives.

First, historical data is selected and grouped according to desired clinical properties, such as primary versus recurrent cancer. The historical data is also grouped according to the chemoresponse grade of the agent administered for treatment (shown are sensitive, intermediate, and resistant chemoresponse grades). Accordingly, the historical data is grouped into six groups, representing: (1) primary cancer and sensitive (S) in vitro efficacy, (2) primary cancer and intermediate (I) in vitro efficacy, (3) primary cancer and resistant (R) in vitro efficacy, (4) recurrent cancer and sensitive (S) in vitro efficacy, (5) recurrent cancer and intermediate (I) in vitro efficacy, and (6) recurrent cancer and resistant (R) in vitro efficacy.

Based upon the outcome of therapy for the members of the groups (e.g., duration of survival or progression-free interval), the groups are each modeled to estimate responses to treatment for patient's that meet the group criteria. For example, the model may be represented by a survival curve, shown in FIG. 9. These models may be compared to show differences in estimated clinical outcome between groups; for example, between group 1 (primary cancer and sensitive in vitro efficacy), and group 2 (primary cancer and resistant in vitro efficacy). Thus, according to the illustration in FIG. 9, a primary cancer patient that receives a drug that tests resistant in culture has an estimated survival of about 30 months (probability of 62%). In contrast, if that same patient were to receive a drug that tests sensitive in culture, the estimated survival duration would be about 55 months (probability of 62%).

The present invention has been described with reference to specific details of particular embodiments thereof. It is not intended that such details be regarded as limitations upon the scope of the invention except insofar as and to the extent that they are included in the accompanying claims. All patents and publications cited are herein incorporated in their entireties for all purposes.

1. A method for estimating chemotherapy outcomes for a cancer patient, comprising:
(A) conducting chemoresponse testing with a panel of chemotherapeutic agents on cultured tumor cells from the patient;
(B) grading the in vitro efficacy for each agent in the panel, wherein a first agent has a first in vitro efficacy against the patient's cells, and a second agent has a second in vitro efficacy against the patient's cells;
(C) estimating an outcome of treatment with said first agent, and estimating an outcome of treatment with said second agent by:
(1) selecting historical outcomes from a database in which a chemotherapeutic agent was administered for treatment after that agent or agent class demonstrated said first in vitro efficacy,
(2) selecting historical outcomes from a database in which a chemotherapeutic agent was administered for treatment after that agent or agent class demonstrated said second in vitro efficacy;
(3) modeling the historical outcomes for said first in vitro efficacy to prepare a first modeled outcome, and modeling the historical outcomes for said second in vitro efficacy to prepare a second modeled outcome; and
(4) comparing the first modeled outcome and the second modeled outcome to thereby estimate chemotherapy outcomes for said first agent and said second agent for said cancer patient.

2. The method of claim 2, wherein the historical outcomes comprise for each cancer subject in a population:
(i) an in vitro efficacy grade on cultured tumor cells for a chemotherapeutic agent received during treatment,
(ii) information on a plurality of clinical variables, and
(iii) an outcome with the chemotherapeutic agent graded in (i).

3. The method of claim 1, wherein the chemotherapy outcome is (i) an objective response, a clinical response, or a pathological response to treatment (ii) survival, progression-free survival, survival after recurrence, or pathological complete response; or a combination of (i) and (ii).

4. (canceled)

5. The method of claim 1, wherein the first modeled outcome and the second modeled outcome are survival curves.

6. The method of claim 1, wherein the first modeled outcome and the second modeled outcome are prepared using a logistic model, Cox Proportional Hazard Model or a Kaplan-Meier Product Limit estimator.

7. The method of claim 1, wherein the historical outcomes are matched to the patient by at least one clinical disease variable.

8. The method of claim 7, wherein the at least one clinical disease variable includes cancer type, cancer stage, cancer grade, tumor histology, tumor debulking status, the presence or absence or level of one or more tumor or serum markers, primary versus recurrent cancer, patient age, investigational site, number and/or type of previous treatments, time since diagnosis, performance status, and extent of remission.

9. The method of claim 8, wherein the clinical disease variables include primary versus recurrent cancer, cancer stage, and status of debulking.

10. The method of claim 1, wherein the chemoresponse testing shows a heterogeneous response across the panel.

11. The method of claim 1, wherein each agent in the panel is tested in the chemoresponse assay at a plurality of concentrations representing a range of expected extracellular fluid concentrations upon therapy.

12. The method of claim 1, wherein said cultured tumor cells are enriched for malignant cells.

13. The method of claim 12, wherein said malignant cells are cultured from explants of the patient tumor specimen.

14. The method of claim 1, wherein said cultured tumor cells are selected from breast, ovarian, colorectal, endometrial, thyroid, nasopharynx, prostate, head and neck, liver, kidney, pancreas, bladder, brain, and lung tumor cells.

15. The method of claim 1, wherein the panel of chemotherapeutic agents comprises at least one agent selected from a platinum-based drug, a taxane, a nitrogen mustard, a kinase inhibitor, a pyrimidine analog, a podophyllotoxin, an anthracycline, a monoclonal antibody, a topoisomerase I inhibitor, bevacizumab, capecitabine, carboplatin, cecetuximab, cisplatin, cyclophosphamide, docetaxel, doxorubicin, epirubicin, erlotinib, etoposide, 5-fluorouracil, gefitinib, gemcitabine, irinotecan, oxaliplatin, paclitaxel, panitumumab, tamoxifen, topotecan, and trastuzumab.

16. (canceled)

17. The method of claim 1, wherein the grading involves determining an adjusted area under curve aAUC for a dose-response curve for each agent in the panel.

18. The method of claim 17, wherein the aAUC accounts for changes in cytotoxicity between dose points along a dose response curve, and assigns weights relative to the degree of changes in cytotoxicity between dose points.

19. The method of claim 18, wherein the changes in cytotoxicity between dose points along the dose-response curve are represented by a local slope, and the local slopes are weighted along the dose-response curve to emphasize cytotoxicity.

20. The method of claim 17, wherein aAUC is determined by:
calculating a Cytotoxity Index (CI) for each dose, where CI=Mean drug/Mean control;
calculating a local slope (S_{d}) at each dose point, where S_{d}=(CI_{d}−CI_{d-1})/Unit of Dose, or S_{d}=(CI_{d-1}−CI_{d})/Unit of Dose;
calculating slope weight at each dose point, where W_{d}=1−S_{d}; and
calculating aAUC, where aAUC=ΣW_{d} CI_{d}, and where, d=1, 2, . . . , 10.

21. The method of claim 17, wherein the levels of sensitivity or resistance to each agent in the panel is determined by comparing each aAUC score to one or more cut-off values.

22. The method of claim 1, wherein (i) said first in vitro efficacy grade is non-responsive and said second in vitro efficacy grade is intermediate responsive; (ii) said first in vitro efficacy grade is responsive, and said second in vitro efficacy grade is intermediate responsive; or (iii) said first in vitro efficacy grade is the highest grade in the panel, and said second in vitro efficacy grade is the second highest grade in the panel.

23.-25. (canceled)
